(19) United States
(12) Plant Patent Application Publication
(10) Pub. No.: US 2005/0273893 P1
(43) Pub. Date: Dec. 8, 2005

(54) Campanula plant named 'Thor Heaven'

(50) CAMPANULA HYBRID; CAMPANULA

(76) Inventor: Jorgen Andersen, Fraugde (DK)

Correspondence Address: JAMES R. CYPHER, 405 14TH STREET, SUITE 1607, OAKLAND, CA, 94612, US

(21) Appl. No.: 10/860,352

(22) Filed: Jun. 3, 2004

(51) Int. Cl. A01H 5/00

(52) U.S. Cl. PLT/263

(57) ABSTRACT

A plant variety of the Campanulaceae family, having the varietal designation ‘Thor Heaven’, with a spreading growth habit and large campanulate, actinomrphic flowers. Leaves are alternate and whorled and fairly small with serrate edges and winged stipules. Mature leaves look like arrowheads. The mature perianth tube is R.H.S. 91 C,D to 92 C (violet-blue group), with dark spots of R.H.S. 71 A (red-purple group) on the tips of the lobes of the perianth.

### LATIN NAME OF GENUS AND SPECIES OF THE PLANT CLAIMED

The new plant is a species of the Campanula genus.

### VARIETAL DENOMINATION

The new plant's varietal denomination is ‘Thor Heaven’.

### BACKGROUND OF THE INVENTION

The present invention relates to a new and distinct variety of the Campanulaceae family. The new variety is named ‘Thor Heaven’. The inventor is Jørgen Andersen, a citizen of Denmark.

### SUMMARY OF THE INVENTION

The distinguishing characteristics of the new variety are retained by asexually reproduced, successive generations.

The plant was developed by the inventor through controlled crosses.

The new variety possesses the commercially desirable characteristics of:
1. a spreading growth habit with dense foliage, and
2. producing many large flowers that at maturity are R.H.S. 91 C,D to 92 C (violet-blue group).

The inventor has asexually reproduced the new variety through three successive generations by cuttings, and has found that the combination of characteristics as herein disclosed remain firmly fixed.

### BRIEF DESCRIPTION OF THE DRAWING

The accompanying drawings serve, by color photographic means, to illustrate the new plant variety. The colors are represented as truly as possible using conventional photographic procedures.

FIG. 1 is a color photograph of a plant grown from a cutting in one pot of the new variety illustrating the overall appearance and form of the plant, and the abundance of blooms.

FIG. 2 is a color photograph of a single branch of the new variety.

FIG. 3 is a color photograph of a leaf of the new variety.

FIG. 4 is a color photograph of the base of the new variety.

FIG. 5 is a color photograph of the plant showing a branch of the new variety.

FIG. 6 is a color photograph of young leaves of the new variety.

FIG. 7 is a color photograph of a number of flowers of the new variety.

FIG. 8 is a color photograph of a number of flowers of the new variety.

FIG. 9 is a view of a single flower of the new variety.

FIG. 10 is a view of a single flower of the new variety.

FIG. 11 is a bottom view of a single flower of the new variety.

FIG. 12 is a side view of a single flower of the new variety.

FIG. 13 is a side view of a single flower with a portion of the corrola tube cut away.

FIG. 14 is a side view with a portion of the corrola tube cut away and with the ovary section as well.

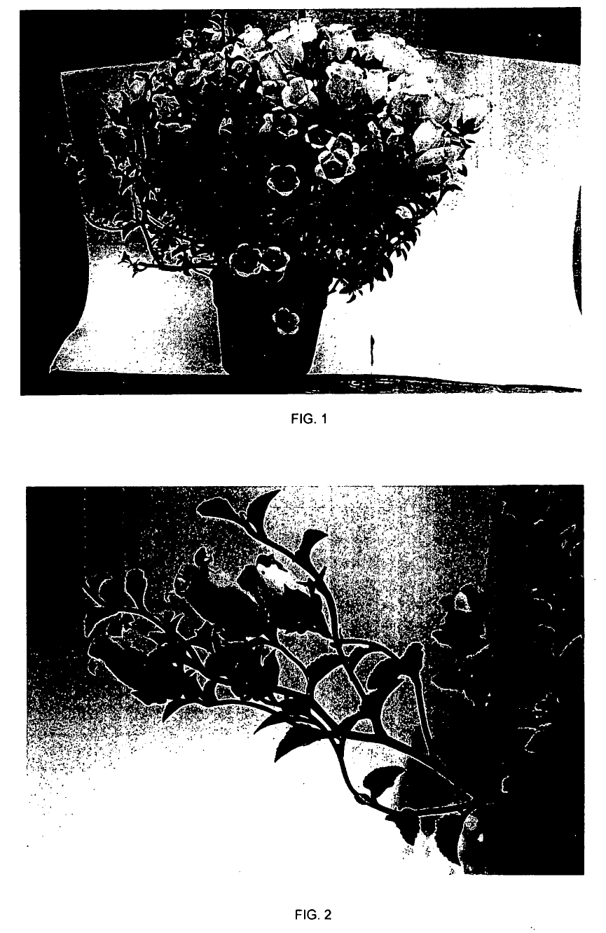

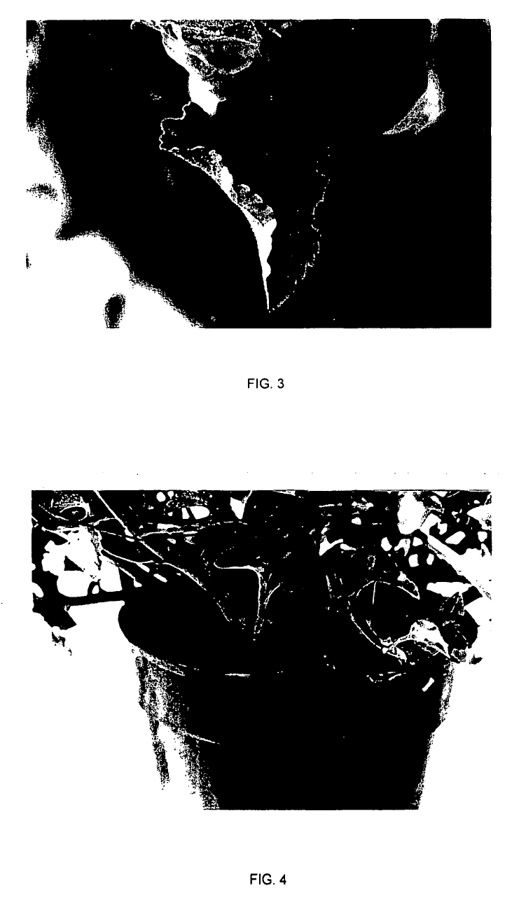

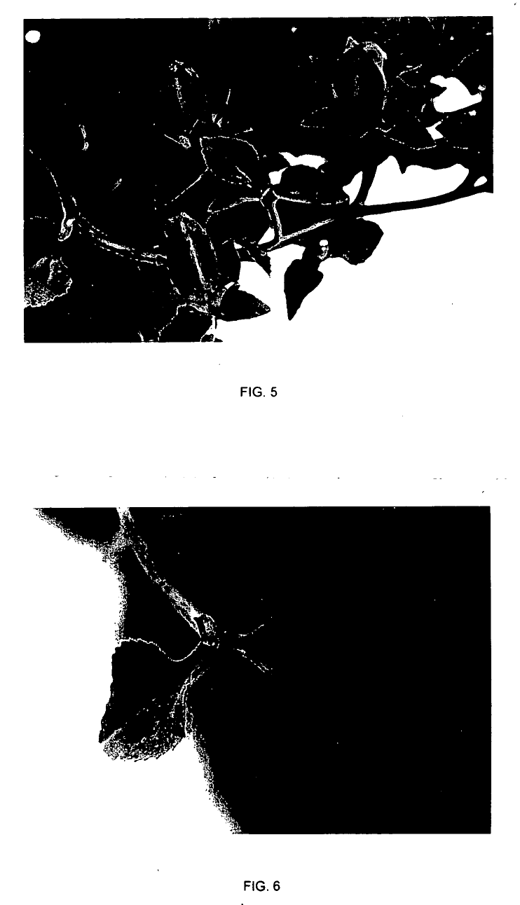

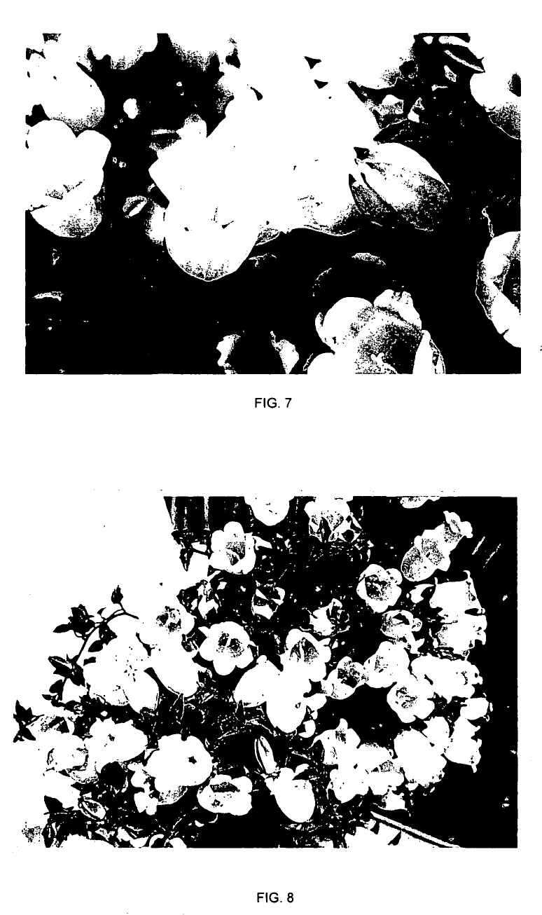

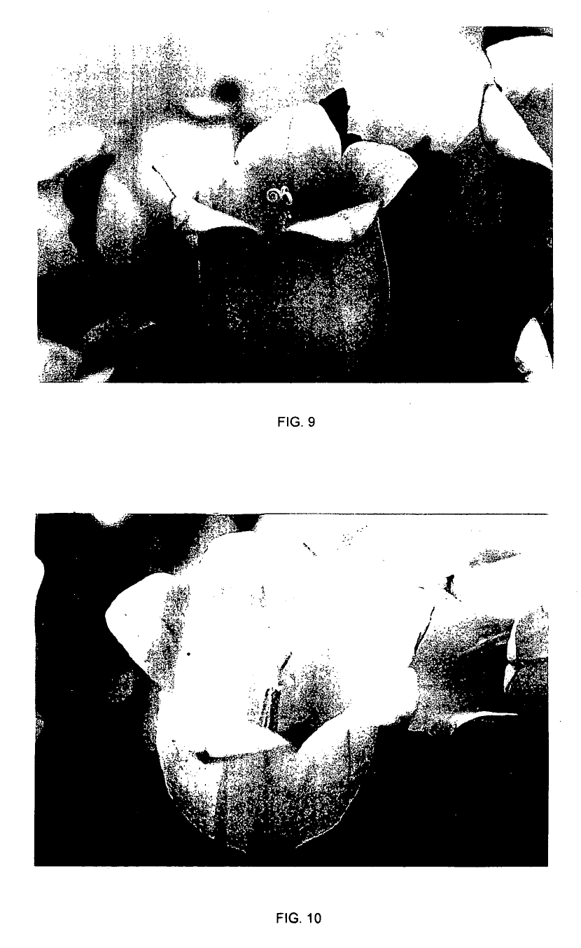

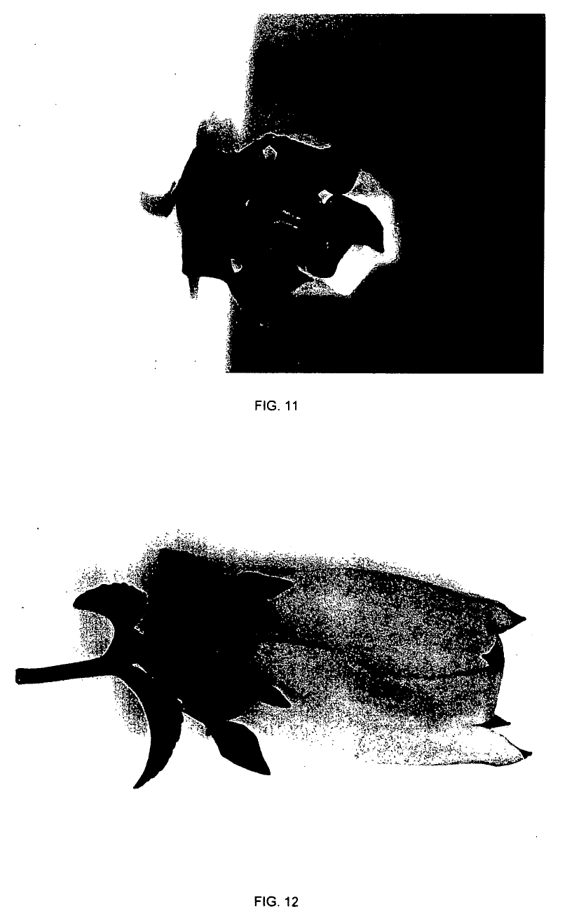

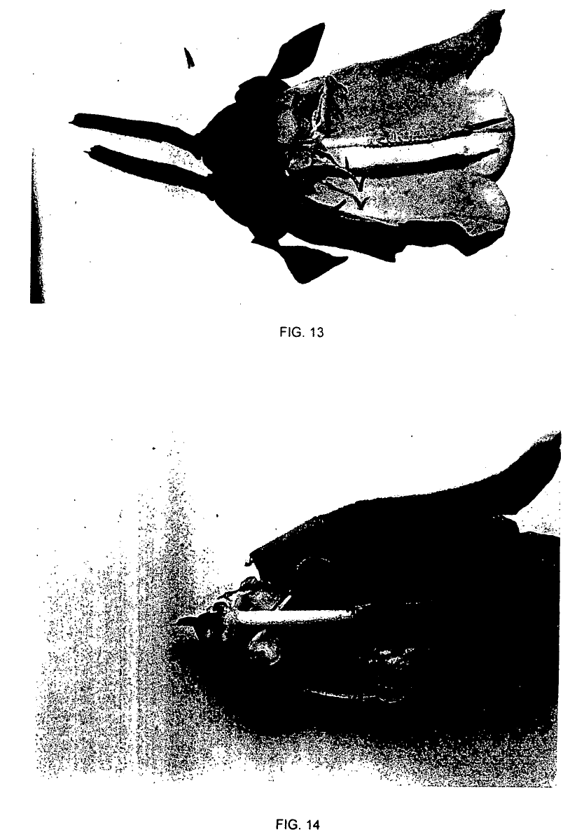

### DETAILED DESCRIPTION OF THE NEW VARIETY

The following is a detailed description of the new variety. The new variety has not been observed under all possible environmental conditions. Color designation and other values stated may deviate slightly from the stated values from flowering to flowering, but the deviations will be within the range expected from varying environmental, seasonal and cultural conditions. Color designations were made according to the R.H.S. Color Chart published by The Royal Horticultural Society of London, England.

The plants at the time of the observation were being grown in 6 inch pots.

The plants had started as lateral cuttings from a plant that had been allowed to grow and then had been cut back to only 1 inch above the soil. The lateral cuttings were taken from the new side shoots that grew from the base stem after it had been cut back.

The cuttings were placed in cells and placed in a fog shower under glass where temperatures ranged from 70 to 80 degrees Fahrenheit, and under dark shade.

The cuttings were fully rooted in approximately 4 to 6 weeks.

The cuttings were then transplanted into 6″ pots, one cutting per pot. After being transplanted, the plants remained inside for 4 more weeks and were then moved outside.

The observed plants were approximately 14 months old.

The observed plants had not been pinched back or pruned.

The following description is based on observations of optimally fertilized plants grown at a nursery in Half Moon Bay, Calif. Temperatures in Half Moon Bay on average range from 55 to 65 degrees Fahrenheit in the summer months, and from 45 to 55 degrees Fahrenheit in the winter months.

### DETAILED PLANT DESCRIPTION

Varietal name: ‘Thor Heaven’.
Classification:
   Family.—Campanulaceae.
   Genus.—Campanula.
Form: Terrestrial plant with many spreading lateral stems. Lateral stems are many branched with the plant spreading well away from its pot.
Height of plants: 14 month old plants are approximately 30 cm high.
Stems:
   General.—Stems are many branched with new shoots growing in leaf interfaces. Lower portions of the lateral stems have stems growing from each leaf node, with no branching below a flower for approximately 5 to 7 leaf nodes. Size: Width — 1 mm at base of flowers and as much as 4 mm wide where lateral stems have grown from original cutting. Color: Predominately R.H.S. 138 B (yellow-green group). Texture — Puberlent.
Leaves:
   General.—Leaves are whorled and alternately arranged on the stem. Very young leaves are entire, and older leaves, farther away from the merristem, becoming very serrate and folded. Mature leaves have acute apexes, sagittate bases and winged petioles, giving the appearance of an arrowhead. Pinnately veined. Size: Length — Mature leaves are approximately 30 mm long including the petiole. Width — Mature leaves are approximately 15 mm wide. Color: Adaxial surface of leaf is predominately R.H.S. 137 D (yellow-green group), while abaxial surface of leaf is predominantly R.H.S. 138 B (yellow-green group). Texture — Abaxial surfaces of leaves are puberlent. Adaxial surfaces of the leaves are puberlent to tomentose.
Flowers:
   General.—Flowers are actinomorphic, solitary, perfect, and 5-lobed. The calyx is united and lobed. The corrola is campanulate. The stamens have winged bases and uniformly deposit their pollen along much of the length of the style below the stigma.
   Sepals.—General: The calyx is 5-lobed, regular and united to the ovary with triangular lobes in each sinus between the sepals. Shape: The apex of the sepals are acute and serrate, and the base is lobed between the sinuses. Size: Length — 15 mm from base of petals to tip of sepals when corrolla is 25 mm long. 20 mm from base of petals to tip of the sepals when the flower is fully mature. When the flower is fully mature the basal appendages are 10 mm long. Width — 10 mm. Color: R.H.S. 137 D (yellow-green group)
   Petals.—General: Petals inserted on ovary and united to form 5-lobed bell-shaped tube. Texture: Succulent. Number: 5. Size (at full bloom): Length of tube — 50 mm. Diameter of tube — 40 mm. Color: The mature perianth tube is R.H.S. 91 C,D to 92 C (violet-blue group), with dark spots of R.H.S. 71 A (red-purple group0 on the tips of the lobes of the perianth. The perianth tube of immature flowers that are just beginning to open is R.H.S. 145 D (yellow-green group).
   Androecium (stamens).—General: 5 inserted stamens having basal wings that cover the ovary, short filaments and long anthers, initially standing erect, but becoming dessicated and highly reflexed above their wings as the flower ages, and depositing their pollen on the style just below the stigma before the flower fully matures. Filament: Shape — Winged at base, with short hairs on the wings. Texture — Glabrous. Color — White. Length — 5 mm. Anther: Shape — Rod shaped. Color — Green when young, but yellowing as it ages and releases its pollen. Size — 11 mm long.
   Gynoecium (pistil).—General: Compound, parietal placentation with united style surrounded by an annular diffuse nectary at its insertion. Style: General — Stout and inserted in ovary. Shape — Elongated. Texture — Fleshy and puberlent just below stigma. Size (at full bloom) — Up to 60 mm long. Stigma: General — Exserted at maturity and erect, usually with 3 lobes that become reflexed as flower ages. Size — 2-3 mm long. Ovary: General — Inferior. Thin epidermis with 3 locules, forming 3 carpels with axile placentation. The ovary is covered by the lobes of the sepals.
   Blooming period.—The plant normally blooms outside in Half Moon Bay, Calif. in May.

1. A new and distinct variety of Campanula plant, substantially as herein shown and described.
